(19) United States
(12) Patent Application Publication
(10) Pub. No.: US 2024/0099628 A1
(43) Pub. Date: Mar. 28, 2024

(54) SALINE ELECTRODE CAP FOR LONG-TERM ELECTROENCEPHALOGRAPH MONITORING AND LONG-TERM ELECTROENCEPHALOGRAPH MONITORING METHOD

(71) Applicant: Qingdao Tenocom Medical Technology CO., LTD., Qingdao (CN)

(72) Inventor: Peng XU, QINGDAO (CN)

(21) Appl. No.: 18/116,242

(22) Filed: Mar. 1, 2023

(30) Foreign Application Priority Data
Sep. 28, 2022 (CN) 202211189396.X
Sep. 28, 2022 (CN) 202222581042.1

(51) Int. Cl. A61B 5/256; A61B 5/00; A61B 5/273; A61B 5/291

(52) U.S. Cl. CPC A61B 5/256; A61B 5/273; A61B 5/291; A61B 5/6814; A61B 2562/046

(57) ABSTRACT

The present application relates to a saline electrode cap for long-term EEG monitoring; the saline electrode cap comprises a cap body, electrode sets, and electrode conductive mediums, the cap body is provided with a plurality of electrode set installation holes which are distributed according to the international 10-20 EEG positioning system standard, the electrode sets are installed in electrode set installation holes; the electrode conductive mediums are arranged in electrode sets and is in contact with a scalp of a subject; buffer members are installed at an end of electrode sets where the electrode conductive medium is installed and are in contact with the scalp of the subject. The saline electrode cap of the present application can reduce the squeezing degree of the electrode cap and a head of the subject on the electrode conductive mediums, the electrode conductive mediums can be prevented from rapidly losing water due to squeezing.

### CROSS-REFERENCE TO RELATED APPLICATION

This application claims the priority benefit of Chinese application serial No. CN202211189396.X filed on Sep. 28, 2022, and Chinese application serial No. CN 202222581042.1 filed on Sep. 28, 2022, the contents of the above identified applications are hereby incorporated herein by reference in its entirety.

### TECHNICAL FIELD

The present application relates to electroencephalograph monitoring technology, and particularly relates to a saline electrode cap for long-term EEG monitoring and a method for long-term electroencephalograph monitoring.

### BACKGROUND OF THE PRESENT INVENTION

Electroencephalographs (EEG) are waveforms of brain electrical signals monitored and recorded by sticking several electrodes onto a scalp according to rules, and are important information for the research and diagnosis of current mental diseases. In most hospitals in domestic, the routine EEG examination time is usually only 20-30 minutes, rarely including the records of sleep stages and episodic events, so its diagnostic value is greatly limited. With the advancement of science and technology, the application of long-term video EGG monitoring capable of recording videos while EGG monitoring is more and more extensive in EGG diagnosis, and software in the long-term video EGG monitoring allows EGG and a video image at each moment to be in one-to-one correspondence, so that a doctor can watch a synchronous video of a subject while watching the EGG, and the accuracy of the examination result is greatly improved.

During EEG monitoring, placing positions of electrodes on a head of the subject are usually according to a international 10-20 system (each electrode is 10% or 20% of a total distance away from the adjacent electrode), which requires medical technicians to measure, calculate and position at the head of the subject in order to install the electrodes accurately. The traditional measurement positioning generally requires two medical technicians to cooperate with each other, the fixing way of electrodes is relatively cumbersome, and the positioning, installing and subsequent disassembly of the electrodes result in a waste of manpower and time. Chinese patent application CN107582052A discloses an electrode cap, wherein a plurality of electrodes are installed on a cap body of the electrode cap, and the electrodes are distributed according to the international 10-20 system and are in contact with the scalp of the subject by means of a sponge soaked with saline, thereby performing EGG monitoring. When the electrode cap is worn on the head of the subject, the sponge soaked with saline easily loses water due to squeezing, which affects monitoring effect; and the sponge will gradually dry up with the increase of monitoring time, resulting in poor conductivity and unstable signals.

In the prior art, there are also electrode caps for EEG detection by coating a conductive paste. Although the electrode caps coated with the conductive paste do not affect monitoring effect due to squeezing and are suitable for long-term monitoring, the gel on the scalp is not easy to remove after monitoring, and the cleaning is very troublesome.

Although Chinese patent application CN113855033A discloses a saline electrode EEG cap easy to assemble and disassemble and capable of flexibly positioning and replenishing, a circle of depressions is arranged at a top of an electrode body, and small hole channels which lead to a bottom sponge core are formed in bottoms of the depressions. During monitoring, the liquid is replenished through small holes to prolong the collocation time.

### SUMMARY OF THE PRESENT INVENTION

The application provides a saline electrode cap for long-term EEG monitoring and a long-term EEG monitoring method.

A first aspect of the application provides a saline electrode cap for long-term EEG monitoring, which is worn on a head of a subject and connected with an EEG monitoring equipment, wherein the saline electrode cap comprises:
a cap body provided with a plurality of electrode set installation holes distributed according to the international 10-20 system, the electrode set installation holes being arranged in a manner of penetrating through the cap body;
a plurality of electrode sets, wherein the electrode sets are installed in the electrode set installation holes, first ends of the electrode sets are located at an outer side of the cap body, and second ends of the electrode sets are located at an inner side of the cap body;
a plurality of electrode slices, wherein the electrode slices are installed on the electrode sets, and the electrode slices are connected with the EGG monitoring equipment through an electrode harness;
a plurality of electrode conductive mediums, wherein the electrode conductive mediums are installed at the second ends of the electrode sets and are in contact with the electrode slices installed on the same electrode sets, and one ends, far away from the electrode slices, of the electrode conductive mediums are in contact with a scalp of a subject; and
a plurality of buffer members which are arranged at the second ends of the electrode sets.

In some of the embodiments, electrode grooves in which the electrode slices are installed are formed in the first ends of the electrode sets; the electrode sets are provided with electrode conductive medium cabins in which the electrode conductive mediums are installed, each electrode conductive medium cabin is provided with a first opening and a second opening, the second openings of the electrode conductive medium cabins are arranged toward the inner side of the cap body so that the electrode conductive mediums are in contact with the scalp of the subject; and the first openings of the electrode conductive medium cabins are communicated with the electrode grooves, so that the electrode slices are in contact with the electrode conductive mediums.

In some of the embodiments, a height of each electrode conductive medium is higher than that of the second opening of each electrode conductive medium cabin, each buffer member surrounds a periphery of the electrode conductive medium cabin, and a height of each buffer member is lower than that of each electrode conductive medium, and is the same as that of the second opening.

In some of the embodiments, a fixed end for fixing the electrode conductive medium is arranged in each electrode conductive medium cabin, and the fixed end is connected to the electrode set.

In some of the embodiments, each electrode conductive medium comprises cotton and a U-shaped sponge, the cotton is close to the first opening of the electrode conductive medium cabin, and is in contact with the electrode slice; and the U-shaped sponge is close to the second opening of the electrode conductive medium cabin, and the U-shaped sponge is in contact with the scalp of the subject and is clamped to the fixed end.

In some of the embodiments, each electrode set is provided with a supplementing hole for supplementing normal saline, and the supplementing hole is located at one side close to the outer side of the cap body and is communicated with the electrode groove.

In some of the embodiments, an insertion direction of each electrode groove is perpendicular to a penetration direction of each electrode conductive medium cabin, one end of each electrode groove along the insertion direction is a closed end, and the other end of each electrode groove along the insertion direction is an electrode insertion hole for allowing insertion of the electrode slice.

In some of the embodiments, a groove is formed in a periphery of each electrode set, and is clamped with the electrode set installation hole.

In some of the embodiments, the electrode harness is connected with the EEG monitoring equipment through a connector, and a female terminal is arranged at one end, far away from the electrode slices, of the electrode harness; the connector is provided with a male terminal, and the female terminal is connected with the male terminal.

In some of the embodiments, the cap body is provided with a plurality of air holes, and the air holes are arranged adjacent to the electrode set installation holes, so that the buffer members can be installed on the electrode sets through the air holes.

A second aspect of the application provides a long-term EEG monitoring method, which adopts the saline electrode cap for long-term EEG monitoring as described above, wherein the method comprises the following steps:
assembling an electrode cap: wherein electrode sets are installed in electrode set installation holes of a cap body; first ends of the electrode sets are located at an outer side of the cap body, and second ends of the electrode sets are located at an inner side of the cap body; and electrode conductive mediums are installed at the second ends of the electrode sets;
soaking the assembled electrode cap in normal saline for a period of time, and then taking it out and wiping off excess water on a surface of the cap body;
wearing the soaked electrode cap on a head of a subject, and allowing the electrode conductive mediums to be in contact with a scalp of the subject;
installing buffer members at the second ends of the electrode sets;
connecting electrode harness with an EEG monitoring equipment for displaying monitoring information; and
installing the electrode slices at the first ends of the electrode sets, and allowing the electrode slices to be in contact with the electrode conductive mediums installed on the same electrode sets.

In some of the embodiments, each electrode set is provided with a supplementing hole for supplementing normal saline, and the supplementing hole is located at the outer side the cap body and is communicated with the electrode groove;
normal saline is supplemented to the electrode conductive mediums through the supplementing holes; by observing an impedance value of each electrode slice on the EEG monitoring equipment, it can be judged whether it is necessary to stop supplementing, the impedance value will gradually decrease with the supplementing of normal saline and finally reach a constant value, and after the impedance value is stable, stop the supplementing of normal saline.

The application also provides a long-term EEG monitoring method, which adopts the saline electrode cap for long-term EEG monitoring as described above, wherein the method comprises the following steps:
S1, installing electrode conductive mediums into electrode sets, then installing the electrode sets into a cap body, soaking the assembled electrode cap in normal saline for 10-15 minutes, and then taking it out and wiping off excess water on a surface of the cap body;
S2, wearing the electrode cap on a head of a subject, and allowing the electrode conductive mediums to be in contact with a scalp of the subject;
S3, installing buffer members at one ends of the electrode sets where the electrode conductive mediums are installed, and allowing the buffer members to be in contact with the scalp of the subject;
S4, correspondingly installing the electrode slices on the electrode sets, and allowing the electrode slices to be in contact with the electrode conductive mediums installed in the electrode sets;
S5, letting the subject lie in a bed and starting long-term EEG monitoring; and
S6: after the monitoring is finished, removing the electrode cap from the head of the subject.

In some of these embodiments, during monitoring process, if the EEG monitoring equipment shows that an impedance value increases abnormally, normal saline is supplement from the supplementing holes, and when the impedance value displayed by the EEG monitoring equipment tends to be stable, supplementing of normal saline is stopped.

Compared with the prior art, the present application has the following beneficial effects: according to the saline electrode cap provided by the present application, the buffer members can increase the contact area between the electrode sets and the scalp of the subject, prevent the electrode conductive mediums from losing water too quickly, alleviate the oppressing sensation of the electrode sets on the head of the subject, and reduce the discomfort caused to the head of the subject. According to the long-term EEG monitoring method provided by the application, the squeezing degree of the electrode cap and the head of the subject on the electrode conductive mediums can be reduced, the electrode conductive mediums can be prevented from rapidly losing water due to squeezing, at the same time the oppressing sensation on the head of the subject can be reduced, the discomfort of the subject in the long-term EEG monitoring process can be reduced, the monitoring method is simple and easy to operate, and the electrode slices are quickly positioned and fixed in a simple way.

### BRIEF DESCRIPTION OF THE DRAWINGS

The accompanying drawings described herein are used to provide a further understanding of the present application and form a part of the present application. The schematic embodiments and their descriptions of the present application are used to explain the invention, and do not constitute an improper limitation of the invention. In the attached drawings:

FIG. 1 is point map of electrode slices according to the international 10-20 system distribution calibrated by the International Federation of Societies for Electroencephalography and Clinical Neurophysiology;

FIG. 2 is a schematic structure diagram of a saline electrode cap for long-term EEG monitoring according to one implementation of the present application;

FIG. 3 is a schematic structure diagram of electrode sets, electrode slices, electrode conductive mediums and buffer members when assembled according to one implementation of the present application;

FIG. 4 is a schematic structure diagram of electrode sets according to one implementation of the present application;

FIG. 5 is a schematic structure diagram of electrode sets, electrode slices, electrode conductive mediums and buffer members when assembled according to another implementation of the present application;

FIG. 6 is a schematic structure diagram of electrode sets according to another implementation of the present application;

FIG. 7 is a schematic structure diagram of electrode sets, buffer members and a cap body when assembled according to one implementation of the present application;

FIG. 8 is a schematic structure diagram of an electrode harness and a connector when assembled according to one implementation of the present application;

FIG. 9 is a schematic structure diagram of a female terminal and a male terminal according to one implementation of the present application;

FIG. 10 is a front view of buffer members according to one implementation of the present application;

FIG. 11 is a top view of buffer members according to one implementation of the present application, in which:

1, cap body; 10, electrode set installation hole; 11, chin strap; 12, air hole; 13, earhole; 2, electrode set; 21, first end of the electrode set; 22, second end of the electrode set; 201, supplementing hole; 202, electrode groove; 221, closed end; 222, electrode insertion hole; 23, fixed end; 24, fixing groove; 25, groove; 26, electrode conductive medium cabin; 260, side wall of the electrode conductive medium cabin; 261, first opening; 262, second opening; 263, first bent part; 264, second bent part; 3, electrode harness; 31, female terminal; 4, connector; 41, male terminal; 5, electrode slice; 6, buffer member; 61, through hole; 7, electrode conductive medium; 71, cotton; 711, first portion; 712, second portion; 72, U-shaped sponge; 73, wet compress cotton; 8, fastening ring.

### DETAILED DESCRIPTION OF THE PRESENT INVENTION

The following is a detailed and complete description for the technical solutions of specific embodiments of the present application in combination with drawings. Apparently, the embodiments are only description for part implementations of the present application, not all implementation of the present application. Based on the general concept of the present application, the embodiments acquired by the person skilled in the art should fall within the protection scope defined by claims of the present application.

In the description of the present application, it is to be noted that the direction or positional relationships indicated by terms “center”, “up”, “down”, “front”, “rear”, “left”, “right”, “vertical”, “horizontal”, “bottom”, “top”, “inner”, “outer” and the like are based on the positional relationships in drawing, these terms are merely used to facilitate the description of the present application and simplify the description, yet do not indicate or imply that the devices or elements referred must have a particular orientation, be constructed and operated in particular orientation, and therefore should not be interpreted as a limitation on this application.

In the description of the present application, it is to be understood that terms “first”, “second” are merely used for descriptive purpose and should not be interpreted to indicate or imply the relative importance or implicitly indicate the number of technical features indicated. Thus, the features defined by “first” and “second” may explicitly or implicitly include one or more of the features.

In the description of the present application, it is to be noted that, unless clearly indicates otherwise, terms “install”, “connect”, “connection” shall be generally understood, such as, may be fixed connection, detachable connection or integrated connection, may be directly connected, or indirectly connected through intermediate media, or inner communication of two components. For the person skilled in the art, the specific meanings of the above terms in the present application may be interpreted according to specific circumstance.

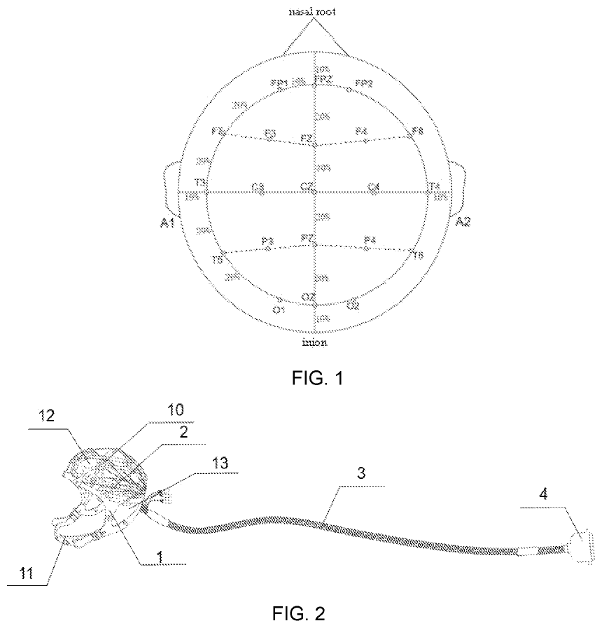

As shown in FIG. 1, a method of positioning electrode slices according to the international 10-20 system is as follows:

Anterior-posterior positions: An anterior-posterior connecting line from the nasal root to the inion (Iz) is divided into 10 equal parts, and a first 10% of a total distance of the connecting line is measured from the nasal root upwards, which point is a frontal pole middle point FPz, and a last 10% of the total distance is measured from the inion zero upwards, which point is an midline occipital Oz. By taking 20% of the total distance being a spacing between FPz and Oz, a midline frontal Fz, a midline center Cz and a midline posterior Pz are determined from front to back.

Horizontal position: A connecting line between highest points of left and right auricles are divided into 10 equal parts, and 10% of a total distance of the connecting line is measured upward from left and right earholes respectively, to obtain a left mid-temporal T3 and a right mid-temporal T4; between T3 and T4, by taking 20% of the total distance of the connecting line being a spacing, a left center C3 and a right center C4 are determined respectively, and Cz is at a midpoint of this line.

Lateral position: From FPz to the back, a left connecting line and a right connecting line are formed by connecting to Oz via T3 and T4, respectively. A first 10% of a total distance of the left connecting line is measured from FPz towards the left rear to determine a left frontal pole FP1, and a first 10% of the total distance of the right connecting line is measured towards the right rear to determine a right frontal pole FP2; a first 10% of the total distance is measured from Oz towards the left front to determine a left occiput O1, and a first 10% of the total distance is measured towards the right front to determine a right occiput O2; between FP1 and O1, and between FP2 and O2, a left front oanterior F7, a right front oanterior F8, a left posterior temporal T5, and a right posterior temporal T6 are determined at a spacing of 20% of the total distance. T3 and T4 are respectively located at midpoints of the connecting lines on the two sides.

A left forehead F3 and a right forehead F4 are located at a midpoint of a connecting line between Fz and F7 and at a midpoint of a connecting line between Fz and F8, respectively; and a left parietal P3 and a right parietal P4 are located at a midpoint of a connecting line between Pz and T5 and at a midpoint of a connecting line between Pz and T6, respectively.

The characteristics of the 10-20 system are that the arrangement of electrodes is proportional to a size and shape of a skull, positions of the electrodes are consistent with an anatomical relationship of cerebral cortex, and names of the electrodes are also consistent with anatomical partitions of a brain. According to an electrode placement method of the 10-20 system, eight points are selected at each of left and right, namely, a frontal pole, a frontal, a lateral frontal, a center, a parietal, occipital, a mid-temporal and a posterior temporal, and three points are selected at the midline, namely, a forehead, a center and a parietal, these points together with left and right earlobe reference electrodes are in a total of 21 electrodes placed.

For convenience of description, a side of the saline electrode cap close to the scalp is called an inner side, and a side of the saline electrode cap far away from the scalp is called an outer side, which is opposite to the inner side.

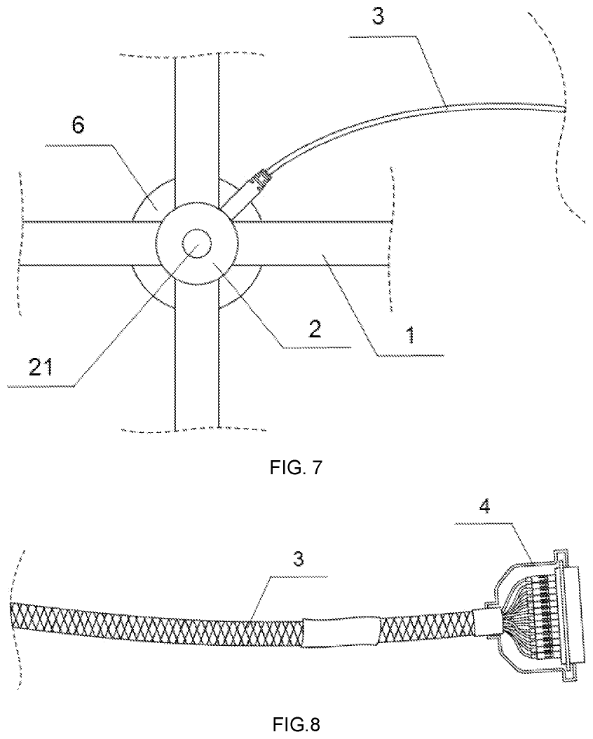

As shown in FIGS. 2-11, in an exemplary embodiment of the saline electrode cap for long-term EEG monitoring of the present application, the saline electrode cap is worn on the head of the subject and connected with the EEG monitoring equipment; the saline electrode cap comprises a cap body 1, electrode sets 2, electrode slices 5, electrode conductive mediums 7 and buffer members 6, wherein the cap body 1 is provided with a plurality of electrode set installation holes 10 which are distributed according to the international 10-20 EEG positioning system standard, and the cap body 1 is a housing structure with a cavity at the inner side. When the subject wears the saline electrode cap, the head of the subject is in contact with a side wall of the cavity, and the electrode set installation holes 10 are formed in a manner of penetrating through the cap body 1. The number of the electrode sets 2 corresponds to the number of the electrode set installation holes 10, and the electrode sets 2 are correspondingly installed in the electrode set installation holes 10. Each of the electrode slices 5 is installed in each electrode set 2, and each electrode slice 5 is connected with the EEG monitoring equipment through an electrode harness 3. Each of the electrode conductive mediums 7 is arranged in each electrode set 2; the electrode conductive medium 7 and the electrode slice 5 installed on the same electrode set 2 are in contact with each other, and one end, far away from the electrode slice 5, of each electrode conductive medium 7 is in contact with the scalp of the subject, so that the electrode slice 5 is connected with the scalp of the subject through saline. Each electrode set 2 is located at the inner side 71 of the cap body, and is sleeved with the buffer member 6. The buffer members 6 can increase a contact area between the electrode sets 2 and the scalp of the subject, alleviate the oppressing sensation of the electrode sets 2 on the head of the subject, and reduce the discomfort caused to the head of the subject.

In view of the softness and elasticity of medical silicone material, the cap body 1 is made of medical silicone material to make the cap body 1 better fit the head of the subject, thus improving the accuracy rate of monitoring and the comfort level of the subject when wearing. The cap body 1 is integrally formed, so as to omit a complicated manual installing process and reduce the production cost; moreover, the cap body 1 can be soaked and cleaned integrally, and is convenient to use. The cap body 1 is provided with a chin strap 11 for fixing the cap body 1, two ends of the chin strap 11 are made of webbing, the chin strap 11 is connected to ear sides of the cap body 1 through buckles or snaps, and the webbing has strong tensile force and wear resistance, so that the friction force and the service life at a connecting part of the chin strap 11 can be increased and prolonged, and the firmness of the chin strap 11 is improved; and a middle part of the chin belt 11 is made of breathable composite fabric, and is provided with a plurality of vent holes so as to facilitate the ventilation of skin. The cap body 1 is also provided with a plurality of air holes 12, the air holes 12 are staggered with the electrode set installation holes 10, so that the electrode sets 2 are installed into the electrode set installation holes and the buffer members 6 are installed at the electrode sets 2, through the air holes 12. Hair of a long-haired subject can be placed on the outer side of the cap body 1 through the air holes 12, so that the electrode slices 5 can be in contact with the scalp of the subject.

The air holes 12 are arranged corresponding to the electrode set installation holes 10, each electrode set installation hole 10 at least has one adjacent air hole 12. In some of the embodiments, the air holes 12 are arranged around the electrode set installation holes 10.

As shown in FIG. 2, earholes 13 are formed in two sides of the cap body 1. The earholes 13 can be used for ears of the subject to extend out, so that the ears are prevented from being pressed by the cap body, and the comfort level of wearing and long-term monitoring can be improved, and at the same time, the earholes can also be used for positioning the cap body when wearing. In some of the embodiments, a position of inion zero (Iz) is marked on the cap body for quick positioning when wearing.

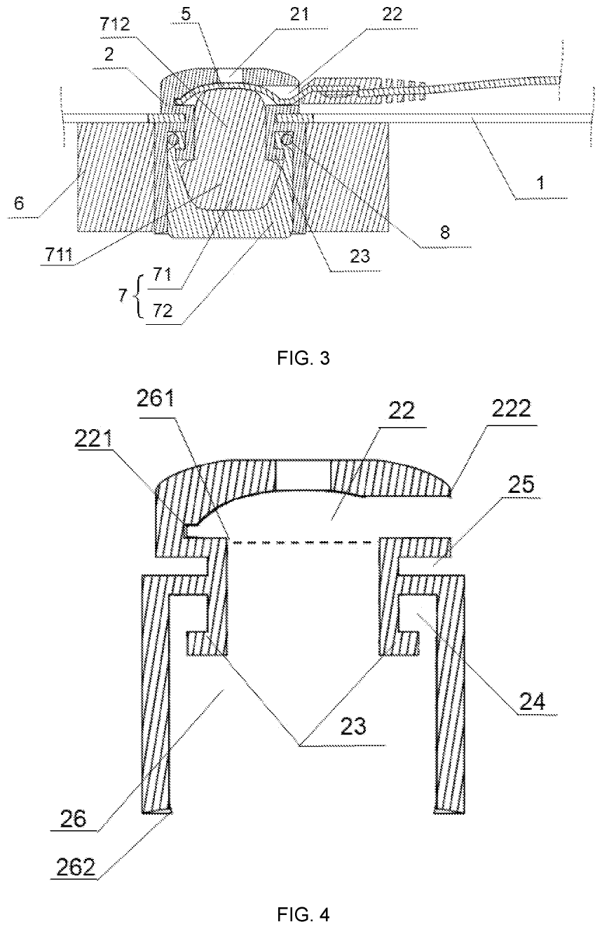

As shown in FIG. 2 to FIG. 7, each electrode set 2 is used for the installing of the electrode slice 5 and the electrode conductive medium 7, a groove 25 is formed at a periphery of the electrode set 2, and the groove 25 is clamped with the electrode set installation hole 10, so that a first end 21 of the electrode set 2 is located at the outer side of the cap body 1, and a second end 22 of the electrode set 2 is located at the inner side of the cap body 1. The cap body 1 made of medical silicone material can facilitate the installing and disinstalling of the electrode set 2. A electrode groove 202 in which the electrode slice 5 is installed is arranged at the first end 21 of each electrode set 2, and the electrode slice 5 is plugged into the electrode groove 202, so that the installing and disinstalling of the electrode slice 5 are facilitated. Each electrode set 2 is provided with an electrode conductive medium cabin 26 in which the electrode conductive medium 7 is installed; the electrode conductive medium cabin 26 is located inside the cap body 1; the electrode conductive medium cabin 26 has two openings, namely, a first opening 261 and a second opening 262; the first opening 261 of the electrode conductive medium cabin 26 is communicated with the electrode groove 202, so that the electrode slice is in contact with the electrode conductive medium 7; and the second opening 262 of the electrode conductive medium cabin 26 is arranged toward the inner side of the cap body 1, so that the electrode conductive medium 7 installed in the electrode conductive medium cabin 26 can be in contact with the scalp of the subject. A penetration direction of each electrode conductive medium cabin 26 is from the first opening 261 to the second opening 262 or from the second opening 263 to the first opening 261. Each electrode conductive medium 7 protrudes from the second opening 262 of each electrode conductive medium cabin 26. A height of each electrode conductive medium 7 is higher than that of an outer edge, facing the second opening 262 of the cap body 1, of each electrode conductive medium cabin 26, so that the electrode conductive mediums 7 can be in contact with the scalp of the subject when the subject wears the saline electrode cap. The direction in which the electrode groove 202 allows the electrode slice 5 inserted into the electrode groove 202 is regarded as an insertion direction of the electrode groove. As shown in FIG. 3, the insertion direction of the electrode groove 202 is perpendicular to the penetration direction of the electrode conductive medium cabin 26, a tail end of the electrode groove 202 in the insertion direction is closed, which is called a closed end 221, to prevent the electrode slice 5 from penetrating through the tail end, and a starting end of the electrode groove 202 along the insertion direction is opened, which is called an electrode insertion hole 222, into which the electrode slice 5 is to be inserted. Each electrode slice 5 is inserted into the electrode groove 202 from the electrode insertion hole of the electrode groove 202 in a direction toward the closed end of the electrode groove 202, so that the electrode slice 5 is installed in the electrode set 2. One side of each electrode groove 202 close to the inner side of the cap body 1 is communicated with the electrode conductive medium cabin 26, so that the electrode slice 5 is in contact with the electrode conductive medium 7. A fixed end 23 for fixing the electrode conductive medium 7 is arranged in each electrode conductive medium cabin 26, and the fixed end 23 is connected to the electrode set 2 so as to fix the electrode conductive medium 7.

In some of the embodiments, the electrode set 2 is provided with a supplementing hole 201 for supplementing saline, and the supplementing hole 201 is located at the outer side of the cap body 1 and is communicated with the electrode groove 202, so that the saline supplemented from the supplementing hole 201 can enter the electrode conductive medium cabin 26 through the electrode groove 202 and be absorbed by the electrode conductive medium 7. The electrode set 2 is made of medical silicone material, which is soft, elastic and comfortable. When the electrode slice 5 is plugged in and out and placed in the electrode groove 202, the electrode groove 202 will not cause wear and damage to the electrode slice 5.

As shown in FIG. 3, in some of the embodiments, each electrode conductive medium 7 comprises cotton 71 and a U-shaped sponge 72, wherein the cotton 71 and the U-shaped sponge 72 are sequentially arranged in the electrode conductive medium cabin 26, the cotton 71 is in contact with the electrode slice 5, the U-shaped sponge 72 is located at one side, close to the inner side of the cap body 1, of the cotton 71, for directly being in contact with the scalp of the subject, and the U-shaped sponge 72 is installed on the electrode set 2 through the fixed end 23.

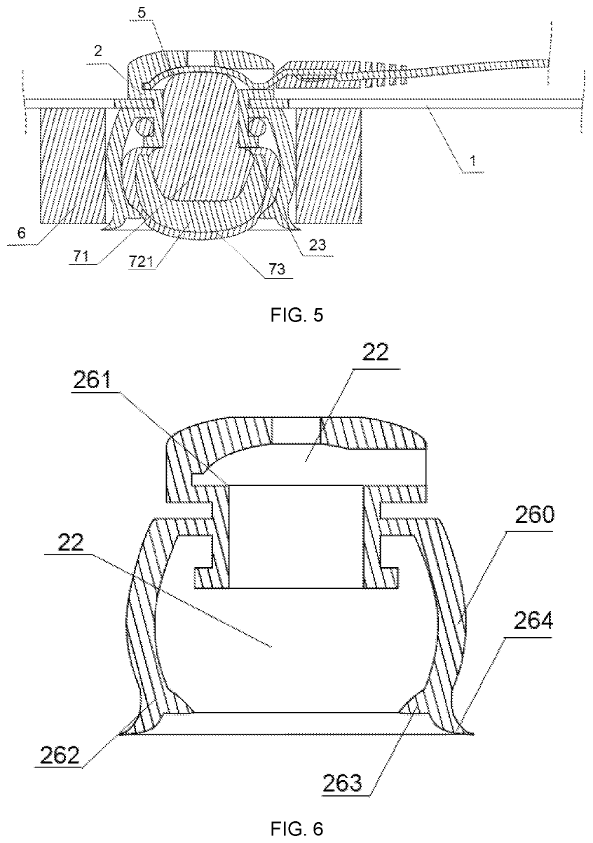

As shown in FIG. 5, in some of these embodiments, each electrode conductive medium 7 comprises cotton 71, an U-shaped sponge 72 wrapped outside the cotton 71, and a wet compress cotton 73 wrapped outside the U-shaped sponge 72. Wet compress cotton is wrapped outside the U-shaped sponge. The addition of wet compress cotton can effectively improve the water absorption and water retention of the electrode conductive medium and the wet compress cotton is soft and has both permeability and wear resistance.

In some of these embodiments, as shown in FIGS. 5 and 6, side wall 260 of the electrode conductive medium cabin is an inward concave arc, that is, the arc protrudes outward, the second opening 262 of the electrode conductive medium cabin has a first bent part 263 bent towards outside of the electrode conductive medium cabin and a second bent part 264 bent towards inside of the electrode conductive medium cabin, and ends of the first bent part 263 and the second bent part 264 facing the first opening are connected with each other 261; the first bent part 263 is located at an inner side of the second bent part 264. Therefore, the first bent part 263 is connected with the wet compress cotton 73, which can make the electrode conductive medium 7 more firmly fixed in the electrode conductive medium cabin 26. The second bent part 264 is close to the scalp, which can effectively prevent the hair of other parts of the subject from contacting with the electrode conductive medium. If the hair contacts with the salt water in the electrode conductive medium, the surface tension of the water will be damaged, and the salt water will be sucked out, affecting its water retention.

The fixed end 23 is an annular structure with a cavity in the middle, and the cotton 71 is clamped in the cavity of the fixed end 23; a fixing groove 24 is formed at an outer side of the fixed end 23; the fixing groove 24 is located in the electrode conductive medium cabin 26; and the U-shaped sponge 72 is connected with the fixing groove 24 through a fastening ring 8. One end of the fixed end 23 faces the first opening 261, another end of the fixed end 23 faces the second opening 262, and the fixing groove 24 is located at the end of the fixed end 23 facing the second opening 262. When the electrode conductive medium 7 is installed at the electrode set 2, the cotton 71 is clamped in the cavity of the fixed end 23, then the cotton 71 is forced to be in contact with the electrode slice 5, the U-shaped sponge 72 coats the periphery of one end, where the fixing groove 24 is arranged, of the fixed end 23, the U-shaped sponge 72 is clamped with the fixing groove 24 through the fastening ring 8, and a closed end of the U-shaped sponge 72 protrudes from the electrode conductive medium cabin 26 and can be in contact with the scalp of the subject. By arranging the fastening ring 8, the cotton 71 and the U-shaped sponge 72 can be more firmly installed at the electrode set 2, the firmness of the cotton and the U-shaped sponge cannot be affected after being soaked in saline for many times, and the replacement of the U-shaped sponge 72 is facilitated. The sponge 72 is U-shaped, and the U-shaped sponge 72 coats the periphery of the fixing groove 24 to limit and block the cotton 71, so as to prevent the cotton 71 from falling off from the electrode conductive medium cabin 26. An outer wall of the U-shaped sponge 72 is clung to a side wall of the electrode conductive medium cabin 26 and is slightly higher than an outer edge of an electrode insertion hole of the electrode conductive medium cabin 26 facing toward the inner side of the cap body 1, with the purpose of reducing the exposed area of the U-shaped sponge 72, thereby reducing a water loss rate of the U-shaped sponge 72. The cotton 71 has strong water absorption, and the U-shaped sponge 72 not only has excellent water absorption, but also has good elasticity, and is not prone to crack and deformation. By combining the cotton 71 with the U-shaped sponge 72, the water retention can be improved as much as possible while keeping the comfort level of the subject. The cotton 71 is divided into a first portion 711 and a second portion 712, the first portion 711 is close to the inner side of the cap body, and an outer contour of the first portion 711 is larger than an inner contour of the fixed end 23, so that the first portion 711 is limited by the fixed end to prevent a situation that the cotton shifts too much when being squeezed and thus the transfer of saline between the cotton and the U-shaped sponge is affected.

As shown in FIGS. 10 and 11, the buffer members 6 are used to increase the contact area between the electrode sets 2 and the scalp of the subject and alleviate the pressure of the saline electrode cap on the scalp of the subject. When the subject wears the saline electrode cap, if only the electrode sets 2 and the electrode conductive mediums 7 installed in the electrode sets 2 are in contact with the scalp of the subject, and the contact area is small, so that the weights of the cap body 1, the electrode slice 5 and other components are applied to the scalp of the subject through the electrode sets 2 and the electrode conductive mediums 7, and the electrode conductive mediums 7 will be compressed when stressed. If the saline electrode cap of this embodiment is not provided with the buffer members, its weight is applied to the scalp of the subject through the second openings of the electrode conductive medium cabins 26, so that a great oppressing sensation is generated on the scalp of the subject, causing discomfort to the head of the subject. The buffer member 6 is an annular structure with a through hole 61 in the middle, the buffer member 6 surrounds a periphery of the electrode conductive medium cabin 26, a height of each buffer member 6 is the same as that of the second opening of each electrode conductive medium cabin 26 and is lower than that of each electrode conductive medium 7, so that the buffer member 6 can exert a function of buffering the pressure on the head of the subject by the buffer electrode set 2 without affecting the contact between the electrode conductive medium 7 and the scalp of the subject. The buffer member 6 is made of a non-bibulous elastic material. When the subject is subjected to EEG monitoring, the electrode cap is worn on the head of the subject, and the U-shaped sponges 72 will lose water due to squeezing. With the support of the buffer members 6, the squeezing degree of the cap body 1 and the head of the subject on the U-shaped sponges 72 can be reduced, and the U-shaped sponges 72 can be prevented from losing water too much due to excessive squeezing. That is, on the premise of not hindering the U-shaped sponges 72 from being in contact with the scalp of the subject, the buffer members 6 can prevent the U-shaped sponges 72 from losing water quickly due to squeezing, and at the same time, can increase the contact area between the electrode sets 2 and the scalp, alleviate the oppressing sensation on the head of the subject, and improve the comfort level of the subject during long-term monitoring.

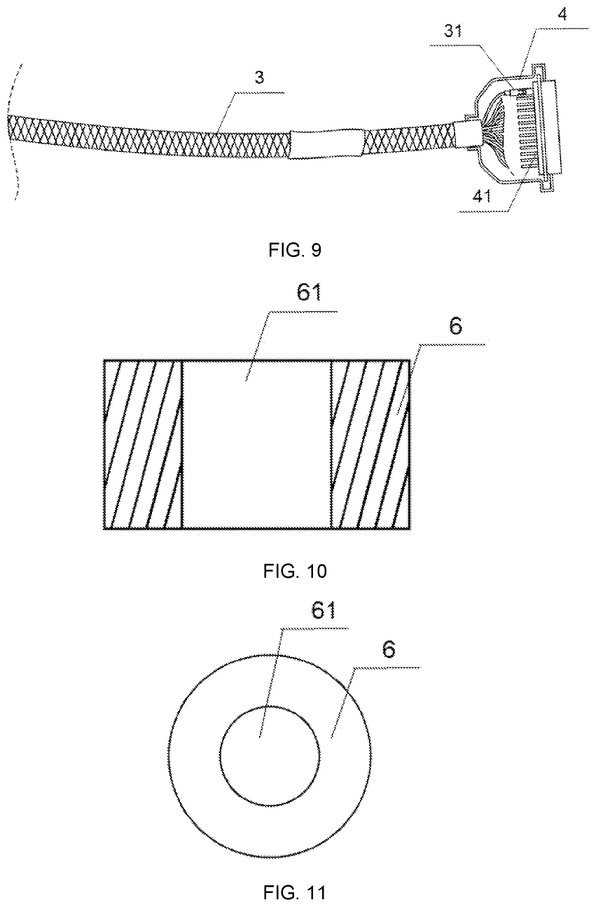

As shown in FIG. 8 to FIG. 9, the electrode harness 3 is used to connect the electrode slices 5 with the EEG monitoring equipment, wherein the electrode harness 3 is connected with the EEG monitoring equipment through a connector 4; the electrode harness 3 comprises several electrode wires, each electrode slice 5 is correspondingly connected with one electrode wire, one end of the electrode wire far away from the electrode slice 5 is connected with a female terminal 31, the connector 4 is provided with several male terminals 41, the male terminals 41 are connected with the female terminals 31 in one-to-one correspondence, and the electrode wires are connected with the connector 4 through the connection between the male terminals 41 and the female terminals 31. The male terminals 41 are plug pins that can be plugged with the female terminals 31, so as to facilitate the connection between the male terminals 41 and the female terminals 31. The electrode slice 5 is a metal electrode slice, which is connected to the electrode wire by welding or crimping, and the connecting part is protected by injection molding, so that the firmness of the connecting part between each electrode slice 5 and each electrode wire can be improved and waterproofing can be achieved effectively. All the electrode wires are fixed into a bundle by using a cable tie or a sleeve net, so as to prevent the monitoring effect from being affected by the winding and knotting of the wire harness, and it is convenient for operators to use. When one of the electrode slices 5 or electrode wires is damaged, the corresponding female terminal 31 can be disassembled and replaced by a single one, so that the time and labor are saved, the maintenance and replacement costs are reduced, and the waste of resources is reduced. By connecting with the EEG monitoring equipment through the connector 4, the electrode harness 3 can be quickly plugged with and unplugged from the EEG monitoring equipment.

The electrode set installation holes distributed according to the international 10-20 EEG positioning system standard are reserved in the cap body 1, and the electrode sets 2 are installed by using the electrode set installation holes to realize the rapid positioning and installing of the electrode slices 5; the electrode slices 5 are connected with the scalp of the subject through the electrode conductive mediums 7, so as to perform EEG monitoring; and buffer members 6 are provided at contact regions between the electrode sets 2 and the scalp of the subject to alleviate the pressure on the scalp of the subject.

As shown in FIGS. 2-9, in an exemplary embodiment of the long-term EEG monitoring method of the present application, the long-term EEG monitoring method comprises the following steps:
assembling an electrode cap: the electrode cap comprises a cap body 1, an electrode sets 2 and an electrode conductive mediums 7, the electrode sets 2 are installed in electrode set installation holes 10 of the cap body 1, and the electrode conductive mediums 7 are installed in the electrode sets 2.

The assembled electrode cap is soaked in normal saline for a period of time and then taken out and excess water on a surface of cap body 1 is wiped off. It should be noted that each electrode conductive medium 7 is in contact with the scalp after absorbing normal saline, after the monitoring is finished, the subject does not need to excessively clean the contact portion between the scalp and the electrode conductive medium 7, but only needs to wipe dry, and after the saline evaporates, the scalp will not be sticky and uncomfortable.

The soaked electrode cap is worn on the head of the subject, a position of the cap body 1 on the head of the subject is adjusted, and the electrode conductive mediums 7 are allowed to be in contact with the scalp of the subject. During wearing, sleeve the earholes 13 on two sides of the saline electrode cap to left and right ears of the subject respectively, then a position of the inion zero (Iz) at the back of the cap body is adjusted to a place aligned with the inion zero of the head of the subject, and the left and right sides of the cap body are adjusted to be aligned, so as to facilitate the subsequent positioning of electrode slices.

The buffer member 6 is installed at an end of each electrode set 2 where the electrode conductive medium 7 is installed, so that the electrode cap is at a certain distance from the head of the subject, thereby reducing the pressure of the electrode cap and the head of the subject on the electrode conductive medium 7 and preventing the electrode conductive medium 7 from rapidly losing water due to squeezing. It should be noted that the buffer members 6 can also increase the contact area between the electrode sets 2 and the scalp of the subject, so as to reduce the oppressing sensation of the electrode cap on the head of the subject. It should also be noted that after the electrode cap is worn on the head of the subject, the electrode conductive mediums 7 will be compressed, and after the buffer members 6 are installed, the buffer members 6 will allow the cap body 1 to have a certain distance from the head of the subject to prevent the electrode conductive mediums 7 from being further compressed.

Electrode slices 5 are connected with the EEG monitoring equipment for displaying monitoring information.

Each electrode slice 5 is installed at one end, facing an outer side of the cap body 1, of the electrode set 2, and each electrode slice 5 is brought into contact with the electrode conductive medium 7 installed in the electrode set 2.

It should be noted that, after a period of monitoring, the electrode conductive medium 7 is likely to dry up and lose water, which will affect the monitoring effect of the electrode slice 5. Therefore, the electrode conductive medium 7 can be supplemented with normal saline through the supplementing hole 201, formed in the outer side of the cap body, of the electrode set 2, so as to maintain the water content of the electrode conductive medium 7. By communicating the supplementing hole 201 with the electrode groove 202 and communicating the electrode groove 202 with the electrode conductive medium cabin 26, the normal saline supplemented from the supplementing hole 201 can enter the electrode conductive medium cabin 26 through the electrode groove 202 and be sucked by the electrode conductive medium 7. Each electrode slice 5 on the EEG monitoring equipment displays an impedance value, when the water of the electrode conductive medium 7 decreases, the corresponding impedance value will increase. With the supplementing of normal saline, the impedance value will gradually decrease, and finally reach a certain stable value. When the normal saline is supplemented to the electrode conductive medium 7, the impedance value of each electrode slice 5 on the EGG monitoring device needs to be observed, and the supplementing is stopped after the impedance value is stable, so that the excessive normal saline supplement is prevented.

As shown in FIG. 2, the electrode slices 5 are connected with the EEG monitoring equipment through an electrode harness 3; when the electrode slices 5 are installed, whether the electrode slices 5 are faulty or not can be judged by the EEG information displayed by the EEG monitoring equipment; and after the electrode slices 5 are installed, the electrode slices 5 are faulty if the EEG monitoring equipment displays no information or displays information abnormally. The EEG monitoring equipment is used to generate an electroencephalogram and related videos, and the information displayed by the EEG monitoring equipment comprises: impedance values corresponding to the electrode slices 5 and a waveform curve including several numerical values. When the EEG monitoring equipment does not display numerical values or the numerical values are displayed unstably, the reasons are that: in one aspect, the electrode slice may be damaged, in which case it needs to be replaced with a new electrode slice 5, and in the other aspect, it may be caused by the lack of water in the electrode conductive medium 7. But the lack of water in the electrode conductive medium 7 will not cause the numerical value corresponding to only one electrode slice 5 to be not displayed or be unstable, so if the numerical value corresponding to only one electrode slice 5 is not displayed or is unstable, the electrode slice 5 may be damaged, and if the numerical values corresponding to multiple electrode slices 5 are not displayed or are unstable, it may be caused by the lack of water in the electrode conductive medium 7. It belongs to the prior art to judge whether the electrode slice 5 is faulty through the information displayed by the EEG monitoring equipment, so it will not be repeated here.

As shown in FIG. 2, the cap body 1 is provided with a plurality of ventilation holes 12. In the step of installing the buffer members 6 at the electrode sets 2, since the electrode cap is worn on the head of the subject and adjusted to a proper position, at this time, the arrangement of the air holes 12 can make the cap body 1 partially turn over, to achieve the effect that on the premise of not disassembling the electrode sets 2, the buffer members 6 can be installed at one ends, facing an inner side of the cap body 1, of the electrode sets 2 without being interfered by the cap body 1. Moreover, for subjects with long hair, they can place their hair on the outer side of the cap body 1 through the air holes 12, so that the electrode slices can be in contact with the scalp.

As shown in FIG. 3, the electrode conductive mediums 7 are used to absorb normal saline, so that the electrode slices 5 can be in contact with the scalp of the subject through normal saline, thereby performing EEG monitoring.

In the above long-term EEG monitoring method, as shown in FIG. 3, FIG. and FIG. 11, when each buffer member is arranged at the inner side of the cap body 1, the fact that the electrode conductive medium 7 is not attached to the scalp is easily caused, so that a situation that the monitoring effect is affected; and since the electrode set 2 often needs to be installed and disinstalled, if the buffer member is provided at the electrode set 2, the installing and disinstalling of the electrode set 2 may be affected. According to the saline electrode cap provided by the present application, the buffer member 6 is designed as an annular structure with a through hole in the middle, and the buffer member 6 surrounds a periphery of the electrode conductive medium cabin 26 of each electrode set 2, so that the buffer member 6 can be installed on or disinstalled from the electrode set 2, and then the buffer member 6 can be installed and disinstalled along with the electrode set 2. A diameter of a middle through hole of each buffer member 6 is the same as that of the periphery of each electrode conductive medium cabin 26, so that the buffer member 6 is closely clung to an outer wall of the electrode conductive medium cabin 26; an outer diameter of the buffer member 6 is twice that of the electrode set 2, and a height of the buffer member 6 is the same as that of an outer edge of an electrode insertion hole, facing the inner side of the cap body 1, of the electrode conductive medium cabin 26, but lower than that of the electrode conductive medium 7, so that the buffer member 6 can buffer the pressure of the electrode set 2 on the head of the subject while not affecting the contact between the electrode conductive medium 7 and the scalp of the subject. The buffer members 6 are made of non-bibulous elastic materials, so that the buffer members 6 can be clamped to the electrode sets 2. When the subject is subjected to EEG monitoring, the electrode cap is worn on the head of the subject, and an U-shaped sponges 72 will be compressed by squeezing. On the premise of not hindering the U-shaped sponges 72 from being in contact the scalp of the subject, the buffer members 6 can prevent the U-shaped sponges 72 from being squeezed excessively and prevent the U-shaped sponges 72 from losing water quickly due to squeezing, and at the same time, the buffer members 6 can also reduce the pressure of the electrode cap on the head of the subject by increasing the contact area between the electrode sets 2 and the scalp, and improve the comfort level of the subject during long-term monitoring.

The above long-term EEG monitoring method will be described in detail in combination with specific embodiments, including the following steps:
S1, installing electrode conductive mediums into electrode sets, then installing the electrode sets into a cap body, soaking the assembled electrode cap in normal saline for 10-15 minutes, and then taking it out and wiping off excess water on a surface of the cap body;
S2, putting the electrode cap on a head of a subject, and allowing the electrode conductive mediums to be in contact with a scalp of the subject;
S3, installing buffer members at one ends of the electrode sets where the electrode conductive mediums are installed, and allowing the buffer members to be in contact with the scalp of the subject;
S4, correspondingly installing the electrode slices on the electrode sets, and allowing the electrode slices to be in contact with the electrode conductive mediums installed in the electrode sets;
S5, letting the subject lie in a bed and starting long-term EEG monitoring; and
S6: after the monitoring is finished, removing the electrode cap from the head of the subject.

In some of these embodiments, during monitoring process, if the EEG monitoring equipment shows that an impedance value increases abnormally, normal saline is supplement from the supplementing holes 201, and when the impedance value displayed by the EEG monitoring equipment tends to be stable, and the supplementing of normal saline is stopped.

The information displayed on the EEG monitoring equipment is observed while the electrode slice 5 is installed, in order to replace the damaged electrode slice 5 in time and avoid affecting the monitoring effect due to using the unqualified electrode slice 5; and it is ensured that all electrode slices 5 are qualified, thereby ensuring the smooth progress of long-term EEG monitoring. It should be noted that, in a practical application, the electrode conductive mediums 7, the electrode sets 2 and the cap body 1 can be assembled into a whole in advance, so that it is not necessary to reassemble every time the EEG monitoring is performed, and it is not necessary to disassemble every time after the EEG monitoring is completed, in order to shorten the monitoring time and improve the monitoring efficiency; even, the electrode conductive mediums 7, the electrode sets 2, the cap body 1 and the electrode slices can be assembled into a whole in advance and soaked in saline together as the electrode slices 5 need to be in contact with saline during monitoring; however, the electrode conductive mediums 7 need to be installed at the electrode sets 2 before being soaked, with the purpose of avoiding a situation that the soaked electrode conductive mediums 7 cannot be installed at the electrode sets 2 due to water absorption and expansion. It should also be noted that brain waves are transmitted to the EGG monitoring device through the electrode slices 5 to generate an electroencephalogram or related video, which belongs to the prior art and will not be repeated here.

In addition, it should be noted that when the U-shaped sponge 72 is damaged and needs to be replaced for the electrode cap assembled in advance, the replacement steps are as follows. Firstly, a damaged electrode set 2 on which a to-be-replaced U-shaped sponge 72 is to be installed is removed together with the U-shaped sponge 72; then, the fastening ring 8 is pulled open and removed, and cotton 71 and the U-shaped sponge 72 can be separated from the electrode set 2 immediately; then, an intact U-shaped sponge 72 is taken, the cotton 71 is put in the electrode conductive medium cabin 26, the U-shaped sponge 72 wraps around the exterior of the cotton 71, and then the fastening ring 8 penetrates through the U-shaped sponge 72 to be clamped into a fixing groove 24 of the fixed end 23, at which time the U-shaped sponge 72 is fixed to the electrode set 2; and finally, a groove 25 of the electrode set 2 is clamped into the electrode set installation hole, thereby completing the replacement of the U-shaped sponge 72. When the electrode slice 5 or the electrode wire is damaged, the replacement steps are as follows. Firstly, a damaged electrode slice 5 (or electrode wire) is taken out of the electrode set 2; then, a housing of the connector 4 is disassembled with a screwdriver tool, the housing of the connector 4 is opened, and the female terminal 31 at the other end corresponding to the damaged electrode slice 5 (or electrode wire) is pulled out of the connector 4; then an intact electrode wire is taken and its female terminal 31 is connected with the corresponding male terminal 41 of the connector 4 in a plug-in manner; and finally, a screwdriver is used to assemble the housing of the connector 4, thereby completing the replacement of the electrode slice 5 (or electrode wire).

According to the long-term EEG monitoring method provided by the above embodiment, the electrode slices 5 are quickly positioned and installed by using the electrode cap, so that the positioning time of the electrode slices 5 in a monitoring process is shortened and a fixing mode of the electrode slices 5 is simplified; the buffer member 6 surrounds the periphery of one end of each electrode set 2 where the electrode conductive medium 7 is installed, so that the oppressing sensation of the electrode cap on the head of the subject is reduced without affecting the contact between the electrode conductive medium 7 and the scalp of the subject; with the arrangement of the fixed end 23 in the electrode conductive medium cabin 26 for fixing the electrode conductive medium 7, the electrode conductive medium 7 can be in close contact with the scalp of the subject with the cooperation of the buffer member 6 and the fixed end 23, and will not lose water quickly due to squeezing, and at the same time, the electrode conductive medium 7 is installed in the electrode conductive medium cabin 26 by the fixed end 23, so that the area of the electrode conductive medium 7 exposed to the air is reduced and the water loss speed is decreased. The supplementing holes 21 are formed in the electrode sets 2 to supplement the normal saline of the electrode conductive mediums 7. The air holes 12 are formed in the cap body 1, so that the electrode sets 2 and the buffer members 6 can be installed conveniently, and the influence of the hair of the long-haired subject on the monitoring results of the electrode slices 5 is reduced. In addition, the cap body 1 is integrally formed by medical silicone material, and is constructed according to a human head so that the cap can better fit the head of the subject, the monitoring accuracy rate is increased and the comfort level of the subject when wearing is improved. The integrally formed cap body 1 is low in cost, is not prone to cracking, can be soaked and cleaned as a whole, and is convenient to use. The electrode set 2 is also made of elastic silica gel, so that the comfort level of the subject when wearing is improved, the replacement of the electrode set 2 is facilitated, and the electrode slices 5 cannot be worn and damaged; by dividing each electrode conductive medium 7 into the cotton 71 and the U-shaped sponge 72, the water retention of the electrode conductive medium can be improved as much as possible on the premise of maintaining the comfort level; and the female terminal 31 is provided at one end of each electrode wire, and the female terminal 31 is connected with the male terminal 41 in a plug-in manner, so that the electrode slice 5 or the electrode wire can be easily replaced.

With the adoption of the long-term EEG monitoring method described above, the pressing force of the electrode cap and the head of the subject on the electrode conductive medium 7 can be reduced, the electrode conductive medium 7 can be prevented from rapidly losing water due to squeezing, and the oppressing sensation on the head of the subject can also be reduced; and the saline can be supplemented to the electrode conductive medium 7 in real time in the monitoring process, so that the EEG monitoring for a long time is facilitated, the monitoring result is accurate, and the monitoring method is simple and easy to operate.

Finally, it should be noted that each embodiment in this specification is described in a progressive manner. Each embodiment focuses on the differences with other embodiments, and the same and similar parts of each embodiment can be referred to each other.

The above embodiments are only used to explain the technical solution of the present application rather than limit it; although the present application has been described in detail with reference to preferred embodiments, those skilled in the art should understand that the specific embodiments of the present application can still be modified or some technical features can be replaced equivalently; without departing from the spirit of the technical solution of the present application, it should be covered in the scope of the technical solution claimed by the present application.

1. A saline electrode cap for long-term EEG monitoring, which is worn on a head of a subject and connected with an EEG monitoring equipment, wherein the saline electrode cap comprises:
a cap body provided with a plurality of electrode set installation holes distributed according to an international 10-20 EEG positioning system standard, the electrode set installation holes being arranged in a manner of penetrating through the cap body;
a plurality of electrode sets, wherein the electrode sets are installed in the electrode set installation holes, first ends of the electrode sets are located at an outer side of the cap body, and second ends of the electrode sets are located at an inner side of the cap body;
a plurality of electrode slices, wherein the electrode slices are installed on the electrode sets, and the electrode slices are connected with the EGG monitoring device through an electrode harness;
a plurality of electrode conductive mediums, wherein the electrode conductive mediums are installed at the second ends of the electrode sets and are in contact with the electrode slices installed on the same electrode sets, and one ends, far away from the electrode slices, of the electrode conductive mediums are in contact with a scalp of a subject; and
a plurality of buffer members which are arranged at the second ends of the electrode sets.

2. The saline electrode cap for long-term EEG monitoring of claim 1, wherein electrode grooves in which the electrode slices are installed are formed in the first ends of the electrode sets; the electrode sets are provided with electrode conductive medium cabins in which the electrode conductive mediums are installed, each electrode conductive medium cabin is provided with a first opening and a second opening, the second openings of the electrode conductive medium cabins are arranged toward the inner side of the cap body so that the electrode conductive mediums are in contact with the scalp of the subject; and the first openings of the electrode conductive medium cabins are communicated with the electrode grooves, so that the electrode slices are in contact with the electrode conductive mediums.

3. The saline electrode cap for long-term EEG monitoring of claim 2, wherein side wall of the electrode conductive medium cabin is an inward concave arc, the second opening of the electrode conductive medium cabin is provided with a first bent part bent towards inside of the electrode conductive medium cabin and a second bent part bent towards outside of the electrode conductive medium cabin, and ends of the first bent part and the second bent part facing the first opening are connected with each other; the first bent part is located at an inner side of the second bent part.

4. The saline electrode cap for long-term EEG monitoring of claim 2, wherein a height of each electrode conductive medium is higher than that of the second opening of each electrode conductive medium cabin, each buffer member surrounds a periphery of the electrode conductive medium cabin, and a height of each buffer member is lower than that of each electrode conductive medium, and is the same as that of the second opening.

5. The saline electrode cap for long-term EEG monitoring of claim 2, wherein a fixed end for fixing the electrode conductive medium is arranged in each electrode conductive medium cabin and the fixed end is connected to the electrode set.

6. The saline electrode cap for long-term EEG monitoring of claim 5, wherein each electrode conductive medium comprises cotton and a U-shaped sponge, the cotton is close to the first opening of the electrode conductive medium cabin, and is in contact with the electrode slice; and the U-shaped sponge is close to the second opening of the electrode conductive medium cabin, and the U-shaped sponge is in contact with the scalp of the subject and is clamped to the fixed end.

7. The saline electrode cap for long-term EEG monitoring of claim 2, wherein each electrode set is provided with a supplementing hole for supplementing normal saline, and the supplementing hole is located at one side close to the outer side of the cap body and is communicated with the electrode groove.

8. The saline electrode cap for long-term EEG monitoring of claim 2, wherein an insertion direction of each electrode groove is perpendicular to a penetration direction of each electrode conductive medium cabin, one end of each electrode groove along the insertion direction is a closed end, and the other end of each electrode groove along the insertion direction is an electrode insertion hole for allowing insertion of the electrode slice.

9. The saline electrode cap for long-term EEG monitoring of claim 1, wherein a groove is formed in a periphery of each electrode set, and is clamped with the electrode set installation hole.

10. The saline electrode cap for long-term EEG monitoring of claim 1, wherein the electrode harness is connected with the EEG monitoring equipment through a connector, and a female terminal is arranged at one end, far away from the electrode slices, of the electrode harness; the connector is provided with a male terminal, and the female terminal is connected with the male terminal.

11. The saline electrode cap for long-term EEG monitoring of claim 1, wherein the cap body is provided with a plurality of air holes, and the air holes are arranged adjacent to the electrode set installation holes, so that the buffer members can be installed on the electrode sets through the air holes.

12. A long-term EEG monitoring method, which adopts the saline electrode cap for long-term EEG monitoring of claim 1, wherein the method comprises the following steps:
assembling an electrode cap: wherein electrode sets are installed in electrode set installation holes of a cap body; first ends of the electrode sets are located at an outer side of the cap body, and second ends of the electrode sets are located at an inner side of the cap body; and electrode conductive mediums are installed at the second ends of the electrode sets;
soaking the assembled electrode cap in normal saline for a period of time, and then taking it out and wiping off excess water on a surface of the cap body;
putting the soaked electrode cap on a head of a subject, and allowing the electrode conductive mediums to be in contact with a scalp of the subject;
installing buffer members at the second ends of the electrode sets;
connecting the electrode slices with an EEG monitoring equipment for displaying monitoring information; and
installing the electrode slices at the first ends of the electrode sets, and allowing the electrode slices to be in contact with the electrode conductive mediums installed on the same electrode sets.

13. The long-term EEG monitoring method of claim 12, wherein each electrode set is provided with a supplementing hole for supplementing normal saline, and the supplementing hole is located at the outer side the cap body and is communicated with the electrode groove;
normal saline is supplemented to the electrode conductive mediums through the supplementing holes; by observing an impedance value of each electrode slice on the EEG monitoring equipment, it can be judged whether it is necessary to stop supplementing, the impedance value will gradually decrease with the supplementing of normal saline and finally reach a fixed value, and after the impedance value is stable, stop the supplementing of normal saline.

14. A long-term EEG monitoring method, which adopts the saline electrode cap for long-term EEG monitoring of claim 1, comprising the following steps:
S1, installing electrode conductive mediums into electrode sets, then installing the electrode sets into a cap body, soaking the assembled electrode cap in normal saline for 10-15 minutes, and then taking it out and wiping off excess water on a surface of the cap body;
S2, putting the electrode cap on a head of a subject, and allowing the electrode conductive mediums to be in contact with a scalp of the subject;
S3, installing buffer members at one ends of the electrode sets where the electrode conductive mediums are installed, and allowing the buffer members to be in contact with the scalp of the subject;
S4, correspondingly installing the electrode slices on the electrode sets, and allowing the electrode slices to be in contact with the electrode conductive mediums installed in the electrode sets;
S5, letting the subject lie in a bed and starting long-term EEG monitoring; and
S6: after the monitoring is finished, removing the electrode cap from the head of the subject.

15. The long-term EEG monitoring method of claim 14, wherein during monitoring process, if the EEG monitoring equipment shows that an impedance value increases abnormally, normal saline is supplement from the supplementing holes, and when the impedance value displayed by the EEG monitoring equipment tends to be stable, supplementing of normal saline is stopped.
